(19) United States
(12) Patent Application Publication
(10) Pub. No.: US 2008/0254103 A1
(43) Pub. Date: Oct. 16, 2008

(54) Wound Dressings Comprising a Protein Polymer and a Polyfunctional Spacer

(75) Inventors: Roy Harris, Nottinghamshire (GB); Wael Nasi, Leicestershire (GB)

Correspondence Address: DAVIS WRIGHT TREMAINE LLP/Los Angeles, 865 FIGUEROA STREET, SUITE 2400, LOS ANGELES, CA, 90017-2566, US

(73) Assignee: ADVANCED PROTEIN SYSTEMS LIMITED, London (GB)

(21) Appl. No.: 10/597,584

(22) PCT Filed: Feb. 17, 2005
(86) PCT No.: PCT/GB2005/000566

§ 371 Date: Jul. 31, 2006

(30) Foreign Application Priority Data
Feb. 17, 2004 (GB) 0403406.2

(51) Int. Cl. A61L 15/16; A61K 47/12; A61L 15/32

(52) U.S. Cl. 424/445; 525/54.1; 514/12

(57) ABSTRACT

There is described a method of forming a wound dressing. The method comprises forming a protein polymer by reacting a protein with a polyfunctional spacer, or an activated derivative thereof. The polyfunctional spacer is preferably a polycarboxylic acid, especially a dicarboxylic acid, and protein polymers prepared using such spacers are suitable for a wide range of therapeutic applications, including use as wound dressings, for the delivery of therapeutically active agents to the body and as bioadhesives and sealants.

This invention relates to the field of wound care, and in particular to the formation of protein polymer gels suitable for topical administration as wound dressings. The invention also relates to the field of drug delivery, and in particular to processes and compositions for the delivery of therapeutic agents either intravenously or topically. The invention also describes a process for preparing protein carrier systems for the attachment or inclusion of therapeutic agents for the treatment of disease states, management of bleeding and tissue repair.

The invention relates to the formation of a range of drug delivery vehicles, from soluble small protein polymers to gels, using an easily-performed chemical procedure. The process is simple and scaleable for commercial use.

Soluble polymers can be used to target specific sites in the body and deliver one or more therapeutically active agents, from small drugs to large proteins. Attachment of the active agent to the polymer is preferably by chemical linkage, or by adsorption, or by inclusion of the active agent into the polymer during formation. More than one agent can be delivered on the same polymer.

The invention also relates to the formation of gels suitable for topical administration, eg to external wounds, burns and ulcers amongst other applications. The application can be either as an inclusion to a bandage, or as a dressing, or as a spray or solution applied directly to the skin and allowed to gel. The gels may also be used internally as vehicles for the slow or controlled release of drugs, and may also be used to prevent or inhibit tissue adhesions following surgical procedures, by forming a barrier between adjacent tissue membranes.

This invention also describes the formation of compounds suitable for the coating of surgical implements, eg catheters or stents, and glass or plastic plates for diagnostic (eg ELISA, ELISPOT) or processing purposes, eg for use in the growing of cells, including stem cells.

The invention also describes the formation of a “natural” tissue sealant with or without the addition of haemostatic and/or clotting agents.

The selection of a wound dressing is complex. The choice of suitable dressing for a patient requires careful and accurate assessment of the wound, knowledge of the healing process, and specific knowledge of the properties of the many dressings available on the market. Patient and economic factors must also be taken into account.

Without careful consideration of all the factors, dressing selection is likely to be arbitrary and potentially ineffective.

It is widely accepted that a warm, moist wound environment encourages healing and prevents tissue dehydration and cell death. Most modern wound care products are designed to provide these conditions.

There are several types of wound care dressings available. Among those that are most commonly used are hydrogels, hydrocolloids, alginates, polymer films and polymer foams. Each product type has general characteristics but the construction and therefore performance of each particular brand may vary considerably within a particular product type. No single product is suitable for use in all wound types or at all stages of healing.

The major characteristics of a dressing that determine its suitability for application to a particular type of wound include its conformability to the body (desirable to maintain complete wound closure), fluid and odour absorbing characteristics, handling and adhesive properties, and the presence of antibacterial and haemostatic activity where appropriate. Other factors which may influence product selection include the potential for the dressing to cause sensitivity reactions, the ease of application and removal (important in minimising pain and trauma to wound surface) and the interval between dressing changes. Dressings should not shed particles or fibres that may delay healing or predispose the wound to infection. They should also not contain extractables that may have an adverse effect of cell growth.

Complete packing of a deep wound is important for moist interactive wound healing ensuring a bacterial barrier and decreased infection rates, decreasing moisture loss, and minimising pain. In ensuring that a cavity is completely packed, dressings are often forced into the wound, further damaging the tissue.

Hydrogel wound dressings are particularly useful for burns, ulcers and deep wounds such as pressure sores because, amongst other things, they soothe pain, give a cooling sensation and provide control of wound surface hydration. Unlike many alginate dressings, they do not stick to the wound and can be removed easily without pre-soaking. However, although easy to use, it is often difficult to completely fill a wound cavity with a hydrogel dressing (eg when packing leg ulcers), and so hydrogel dressings often provide a poor barrier against bacteria and may not be suitable for use on infected wounds.

There clearly exists a need for improved wound dressings that exhibit a greater number of desired characteristics, being more “universal”, in that they are suitable for a wider range of wound types and stages of healing.

In particular, a dressing with the benefits of a hydrogel dressing, but superior anti-bacterial properties and the ability to completely fill wound cavities of any shape and size would provide a valuable improvement over current hydrogels.

Furthermore, a wound dressing that also delivers active ingredients, eg drugs to the wound site in a controlled manner would be of additional benefit. Desirable active ingredients may help to fight or protect against infection, reduce pain, reduce inflammation and/or facilitate healing, eg by encouraging clotting.

Human serum albumin (HSA) protein has been found to exhibit a number of properties that make it particularly beneficial for wound healing. For example, by reversibly binding a wide range of drug molecules, HSA may offer a controlled release mechanism for drug delivery. HSA binds metal ions (eg zinc, copper and silver), which may be important in the anti-infective treatment of wounds, and may detoxify the wound site and scavenge free radicals. Pathological platelet aggregation is inhibited by HSA, and inflammatory chemical levels (and therefore itching) are also decreased. HSA is non-allergenic and may naturally confer anti-bacterial/antiviral activity at the wound site.

Albumin is employed for a number of other medical uses, eg to increase blood volume. WO 99/66964 relates to albumin-based compositions for use as bioadhesives, surgical sealants, and implantable devices for drug delivery and prosthesis. The adhesive properties of these compositions make them unsuitable for use as external wound dressings and, although the compositions are intended to break down in the body, suitability for internal use is also limited by unwanted adhesion. Following surgical procedures, an adhesive intended to re-join damaged tissue may also attach the wound site to adjacent tissues/organs and cause further damage.

WO 99/66964 discloses the use of accessory molecules to alter the rate and/or degree of cross-linking between albumin molecules. It is stated that dicarboxylic acids are able to accelerate the gelation of bovine serum albumin. However, we have found that products formed in accordance with WO99/66964 are dry and brittle in comparison to the polymers of the present invention. Such brittle products are unsuitable for use as wound dressings.

There has now been devised a method of forming a wound dressing that overcomes or substantially mitigates the above-mentioned and/or other disadvantages associated with the prior art.

According to a first aspect of the invention there is a method for the formation of a wound dressing, which method comprises forming a protein polymer by reacting a protein with a polyfunctional spacer, or an activated derivative thereof.

The wound dressing may be formed in situ. By “in situ” is meant in the context of the present invention that reaction of the protein with the polyfunctional spacer to form the dressing occurs at the wound site. The components of the composition may be applied to the wound site simultaneously or in quick succession, or the components may be mixed immediately prior to use and the mixture then applied to the wound site.

In situ formation of the wound dressing is particularly advantageous in that the dressing takes on the exact shape of the wound, completely filling the wound cavity without aggravating the exposed tissue. The precise fit ensures that the wound is totally sealed.

Supporting substrates may be incorporated into the dressing in situ by addition to the composition before gelling occurs or during the gelling process. In particular, it may be preferable to cover the composition with a vapour-permeable membrane that will prevent the polymer gel from drying out and, most importantly, keep the wound moist. The vapour-permeable membrane would preferably be added at the end of the gelling process so that it is firmly and evenly attached but does not sink too far into the composition.

The wound dressings of the present invention may also be pre-formed (ie cross-linked before application to the wound site). Such dressings may take the form of bandages impregnated with the protein polymer, or gel sheets, either with or without a supporting substrate. Gels of particular shapes and sizes may be specifically moulded for particular wound types or body areas. Alternatively, appropriately sized dressings may be cut to size from larger gel sheets immediately before application.

By a “protein polymer” is meant in the context of the present invention a polymeric species made up of a plurality of complete protein units linked together by linking groups derived from the polyfunctional spacer. It will be appreciated that an individual protein molecule is “polymeric” in the sense of being made up of a chain of amino acid residues that are covalently bound together. Such an individual protein molecule is not a “protein polymer” within the meaning of that term as used herein. Instead, the protein polymer is the reaction product generated by the coupling together of individual protein molecules to form a chain or matrix of such molecules covalently bound together via linking groups.

Proteins that may be used as in the present invention include globular proteins and fibrous or structural proteins, and mixtures thereof.

Examples of globular proteins include synthetic or natural serum proteins, natural or synthetic derivatives thereof, salts, enzymatically, chemically, or otherwise modified, cleaved, shortened or cross-linked, oxidised or hydrolysed derivatives or subunits thereof. Examples of serum proteins are albumin, α-globulins, β-globulins, γ-globulins, fibrinogen, haemoglobin, thrombin and other coagulation factors. Examples of fibrous or structural proteins include synthetic or natural collagen, elastin, keratin, fibrin, and fibronectin, natural or synthetic derivatives thereof, and mixtures thereof.

Particularly preferred proteins are albumins.

Where the protein polymers prepared in accordance with the invention are intended for administration to the human body, the protein used is preferably of human origin, ie actually derived from humans, or is identical (or substantially so) in structure to protein of human origin. A particularly preferred protein is thus human serum albumin.

Human serum albumin may be serum-derived, for instance obtained from donated blood. Human serum albumin is readily available as a fractionated blood product and has been safely used for many years for intravenous delivery as a blood expander. However, in order to eliminate or reduce the risk of transmission of potential contaminants, eg viral or other harmful agents, that may be present in blood-derived products, as well the potential limitations on supply associated with material isolated from donated blood, the protein, eg human serum albumin, may be a recombinant product derived from microorganisms (including cell lines), transgenic plants or animals that have been transformed or transfected to express the protein.

For veterinary use, non-human animal-derived protein may be used, as appropriate. Examples of such proteins include horse serum albumin, dog serum albumin etc.

Mixtures of proteins, ie more than one different protein, may be used.

Functional groups on the protein molecules with which the spacer may react include amino groups. Preferred proteins therefore include proteins with relatively high proportions of amino acid residues that include free amino groups, particularly NH₂ groups. One example of such an amino acid residue is lysine, and so particularly preferred proteins for use in the invention include proteins including lysine residues, especially proteins with high proportions of lysine residues, eg more than 20 lysine residues per protein molecule, more preferably more than 30 or more than 40 lysine residues.

Polyfunctional spacers that may be used in the present invention include polycarboxylic acids, polyamines, poly(carboxy/amino) compounds (ie compounds having a multiplicity of carboxyl and amino groups), polyalcohols, polyketones, polyaldehydes, and polyesters.

Polycarboxylic acids or polyamine spacers are preferred, more preferably dicarboxylic acids or diamines.

Polycarboxylic acids include citric acid and polyacrylic acid.

Preferred spacers are bifunctional spacers, particularly homobifunctional spacers.

Polyamines include poly(lysine) and chitosan

Particularly preferred spacers are dicarboxylic acids.

The dicarboxylic acid spacer is most preferably an alkylene dicarboxylic acid, particularly a straight-chain alkylene dicarboxylic acid molecule of the formula:

HOOC(CH₂)ₙCOOH

in which n is from 1 to about 20. Preferably n is from 2 to 12, more preferably from 3 to 8.

Preferred straight-chain alkylene dicarboxylic acid spacers are:

| n | Common name | Systematic name | Formula |
|---|---|---|---|
| 2 | Succinic Acid | Butanedioic Acid | HOOC(CH₂)₂COOH |
| 3 | Glutaric Acid | Pentanedioic Acid | HOOC(CH₂)₃COOH |
| 4 | Adipic Acid | Hexanedioic Acid | HOOC(CH₂)₄COOH |
| 5 | Pimelic Acid | Heptanedioic Acid | HOOC(CH₂)₅COOH |
| 6 | Suberic Acid | Octanedioic Acid | HOOC(CH₂)₆COOH |
| 7 | Azelaic Acid | Nonanedioic Acid | HOOC(CH₂)₇COOH |
| 8 | Sebacic Acid | Decanedioic Acid | HOOC(CH₂)₈COOH |

Straight-chain alkylene dicarboxylic acids are particularly useful spacers because the properties of the resulting protein polymers may be varied simply by varying the length of the alkylene chain. In general, at a fixed protein concentration the gelling time decreases and the polymers become harder, less rubbery and more turbid with increasing dicarboxylic acid chain length. The chemistry is simple, yet a wide range of protein polymer systems may be prepared by adjustment of only a small number of variables. As well as promoting a high degree of control, the properties of the polymers can be anticipated reasonably well from the composition and reaction conditions.

In order to facilitate reaction of the spacers with the protein molecules, it will generally be desirable for the spacer to be activated, ie for the functional groups of the spacer to be converted to groups of greater reactivity towards groups in the protein. Suitable activation chemistries will be familiar to those skilled in the art, and include the formation of active ester groups.

One particular class of activators, suitable for use with dicarboxylic acid spacers, is carbodiimide compounds, and a particularly preferred activator for use in the invention is ethyl[dimethylaminopropyl]-carbodiimide (EDC). In one embodiment of the invention the dicarboxylic acid (preferably C6-C10 in length) is added to the protein solution. EDC is added to the mixture and the reaction is allowed to proceed. The concentration of the protein solution, the proportion of dicarboxylic acid to protein, the amount of EDC and the time are all important to the desired result. The EDC activates —COOH groups and allows linking with free amine groups on the protein.

The control of the reaction means that the polymerisation can be controlled to give soluble polymers, insoluble particles or gels from the same reaction mixture. Greater than 95% conversion of the starting protein concentration to a polymer may be obtained, and up to 100% conversion into a gel.

The omission of the dicarboxylic acid spacer, and the use of EDC alone (under the conditions described here), leads only to partial polymerisation over a period of several hours to days, with a lower yield of polymer compared to that obtained when a dicarboxylic acid is used.

In general, the method according to the invention will be carried out in solution. Preferably, an activating agent, eg EDC, is added to a solution of the protein and the dicarboxylic acid. The EDC may be in solution, eg with distilled water, or it may be added to the protein and dicarboxylic acid solution in a solid form, eg powder. Although in principle it is also possible to firstly activate the dicarboxylic acid with EDC and then to add the activated spacer to the protein solution, this has been found in practice not to produce results as good as those obtained by adding EDC to the mixture of protein and spacer.

For ease of application, it may be desirable to formulate the reactants as a mixed dry powder to which water, saline or a buffer solution is added immediately prior to application. The protein and dicarboxylic acid may not react without addition of EDC so, in order to store the reagents as powders without risk of premature reaction, it may be desirable keep the protein/dicarboxylic acid powder separate from the EDC powder, eg by containment in separate sachets. A preferred method of application is a syringe containing a solution of the protein/dicarboxylic acid solution and EDC powder, the solution and the powder being separated by a frangible membrane. By pressing the plunger of the syringe, the user forces the membrane to rupture and the reagents to mix immediately prior to application.

Application of solutions to a wound site may be by pouring, painting or spraying of the solutions.

It may be desirable for a wound dressing to deliver therapeutically active ingredients to the wound site. Drugs such as antibiotics, antivirals, anti-inflammatory agents, haemostatic agents, pain killers and phages may be added directly to the composition or via carriers that promote absorption from the wound site, eg liposomes. Actives that promote or improve tissue repair may also be incorporated, eg growth factors, anti-scarring agents, and agents that promote angiogenesis. By eliminating infection and absorbing exudates, the smell of malodorous wounds can be reduced. However, wound odour may also be reduced/removed by incorporating agents (eg charcoal) into the dressing which absorb the volatile molecules that are responsible for the smell.

The incorporated active compounds will be delivered to the wound site by leaching from the gel and by release from the gel as it degrades. A key factor in determining the rate of release of an active will be the softness/hardness of the protein polymer. Active compounds will leach out of softer polymers more easily because they are not held in as effectively by the cross-linking protein molecules. Softer polymers will also break down at a faster rate because the looser structure will allow moisture and enzymes to penetrate more easily.

According to another aspect of the invention, there is provided a wound dressing prepared by the methods described above, ie a wound dressing comprising a protein polymer formed by reacting a protein with a polyfunctional spacer or an activated derivative thereof.

The particularly preferred chemistry of the present invention has also been found to produce protein polymers that are suitable for a number of other therapeutic applications.

Thus, according to another aspect of the invention, there is provided a method of forming a protein polymer, which method comprises reacting a protein with a dicarboxylic acid or an activated derivative thereof, provided that the protein is not bovine serum albumin.

A further aspect of the invention is a method of forming a protein polymer, which method comprises reacting a protein with an alkylene dicarboxylic acid or an activated derivative thereof.

The protein is preferably an albumin, particularly human serum albumin.

By appropriate choice of reactants and reaction conditions, products with a wide variety of properties can be prepared. Thus, the protein polymers may be prepared in soluble form, in the form of insoluble particles, or in gel form. The gel form can be varied from very sticky to soft but non-adhesive, and the hardness can be incrementally increased up to very hard gels with low deformation. Parameters that can be varied to achieve these differing results include the choice of protein starting material, the choice of spacer, concentrations of the various reactants, the reaction temperature and duration of the various reaction steps.

The speed of gel formation can also be varied over a wide range, from seconds to minutes to hours, by controlling the ratio of reagents used to form the gel and the temperature.

The gelling reaction is best performed at mildly acidic pH (eg pH=5-6). However, it is often preferable to raise the final pH of the gel to close to physiological pH. There are a number of ways of controlling the pH of the final gel. One approach is to vary the molar ratio of protein to dicarboxylic acid; low levels of dicarboxylic acid give gels of close to physiological pH. A second approach is to vary the molar ratio of protein to EDC; high EDC levels result in gels of higher pH. For those skilled in the art it can be seen that it is possible to find a balance of conditions that achieves the required gel consistency for a particular application at the desired pH.

The gelling reaction may be a biphasic reaction where initial gelling is followed by a secondary “curing” stage. The reaction will not proceed to the curing stage for certain combinations of HSA, dicarboxylic acid and EDC, eg if the level of EDC is too low. Instead, a drop in pH is observed after gelling and the gel re-dissolves. It is thought that a minimum percentage of carboxylic acid groups must be activated by the EDC in order to drive the reaction all the way to the curing stage. Polymers with a low pH are generally less stable because of the unreacted carboxylic acid groups present on the spacer and HSA.

The addition of further compounds may be advantageous. For example, the addition of drugs or other active compounds for controlled release (as described in relation to wound dressings above), and/or other modifying agents which alter the properties of the polymer, eg to release water, to affect flexibility, improve absorbance, skin-feel and aesthetics, mechanical and/or adhesive strength or to alter the degradation profile of the protein polymers.

Ethanol, glucose and glycerol are examples of compounds that may be added to the protein gels of the present invention.

Ethanol, a well-known bacteriostat, may be added to improve the anti-bacterial properties of the gel, glucose to provide a source of energy and thereby to promote cell growth, and glycerol to help prevent moisture loss and maintain gel integrity at the wound site.

Glucose may be particularly useful in wound dressings of the present invention for use on chronic wounds because chronic wounds generally have a poor blood supply, hence poor energy supply and therefore poor cell growth.

We have found that the addition of ethanol, glycerol or glucose improves the consistency of the polymers by further reducing brittleness.

Although it is possible to add modifying agents to the HSA and dicarboxylic acid in one step, in practice (using ethanol, glucose, or glycerol) we found that it is more effective to modify a percentage of the HSA with the modifying agent prior to mixing with the remaining unmodified HSA and carboxylic acid spacer. Thus, the modifying agent is added to aqueous HSA, and EDC is added to facilitate the reaction. The modified HSA and ethanol solution is added to a solution of unmodified HSA and dicarboxylic acid, and then this “gelling solution” is reacted with EDC to form a gel.

As well as modification of the protein being used to improve the physical properties of the protein polymer, modified HSA may have utility as a therapeutic. De-liganded albumin, for example, has available binding sites which may trap and remove toxins, cytokines and the like.

Polymers may be prepared in soluble form using low protein concentrations. Soluble polymers are more easily produced at neutral pH. Low concentrations and neutral pH are easily achieved by adding a suitable buffer, eg phosphate buffered saline. Soluble polymers are suitable for parental delivery and have a number of applications as delivery vehicles, eg delivering drugs, delivering contrast agents useful in imaging techniques, or as platelet substitutes or enhancers (delivering haemostatic agents).

The need for platelet substitutes and/or enhancers is being driven by their application in the treatment of cancer patients. One of the side-effects of cancer therapy is the drastic reduction in platelets, or thrombocytopenia. The condition is currently treated with a transfusion of blood-derived platelets, but as chemotherapy regimes become even more aggressive and as the use of bone marrow transplantation increases, the requirement for platelets is growing. Furthermore, blood-derived platelets have the potential to transmit viral infections, suffer from instability during storage, and cause immune reactions.

The terms ‘platelet substitutes’ and ‘platelet enhancers’ are often interchanged, whether incorrectly or for convenience. By ‘platelet substitutes’ in the context of the present invention is meant a complete platelet replacement which does not necessarily require the presence of naturally produced platelets. ‘Platelet enhancers’, on the other hand, may require the natural formation for a platelet plug at the wound site (and so the natural platelet count may need to be above a threshold level). Platelet enhancers then aggregate at the platelet plug to form a clot, thereby improving the activity of platelets in thrombogenic conditions. Platelet substitutes/enhancers may be prepared according to the present invention by immobilising clotting agents or other active peptide derivatives to the surface of the polymer in such as way as to maintain their biochemical activity. In particular, protein polymers of the present invention may be conjugated with such agents that promote or regulate platelet adhesion and aggregation through specific receptors expressed on the platelet surface. An example is the GPIIb/IIIa receptor that interacts with fibrinogen, active peptides of fibrinogen and von Willebrand's factor. Methods of conjugating with fibrinogen include thiolating the protein polymer, activating the fibrinogen with N-[maleimidocaproic acid] hydrazide and then conjugating the activated fibrinogen via the thiol groups on the protein polymer. The platelet substitute/enhancer can be delivered by intravenous infusion and is activated at the site of internal wounds in the blood vessels.

As a delivery vehicle, the protein polymers are suitable for the slow or controlled release of drugs. Furthermore, by delivery of active agents or by virtue of their absorption properties, the protein polymers of the present invention may be useful for detoxification applications.

The protein polymers may naturally enhance drug delivery to areas of the body that are difficult to target independently. More preferably, the protein polymers may be conjugated with one or more targeting moieties that have an affinity with a specific locus in the body. Suitable targeting moieties may be antibodies. An antibody may act as a therapeutic agent in its own right, or else one or more secondary agents may be attached, eg cytotoxics, radionuclides for targeted anticancer therapies, or vaccines or genes. A targeting moiety may have an affinity with a particular organ or site of a disease, it may enhance delivery of the secondary agent to that location, and/or may alter the biodistribution of those agents, for example by causing the agent to accumulate in a particular organ, eg the liver, thereby allowing that organ to be targeted.

Similarly, protein polymers of the present invention may be bound with targeting moiety and a contrast agent. Contrast agents may be metals useful in magnetic resonance imaging (MRI), or in nuclear imaging, or as therapeutic agents in radiotherapy.

Insoluble protein particles can be prepared with increased concentration of dicarboxylic acid spacer relative to the activating agent and/or increased reaction time whilst maintaining a low protein concentration. Alternatively insoluble particles can be produced by dispersing soluble protein polymers of the present invention in organic solvents, eg acetone.

Using the method of the invention, protein polymer gels can be produced with differing consistencies (soft to hard), and differing adhesive strengths.

Non-adhesive protein gels of the present invention are useful in preventing or inhibiting tissue adhesions following surgical procedures by forming a barrier between adjacent tissue membranes. By adjustment of the reagents and reaction conditions, the speed of degradation can be chosen so that, for example, the polymer can be designed to degrade as the wound heals. The in situ formation of the gel will ensure total coverage of a particular area, to a desirable thickness. The gel may be applied as a thin film or else the composition may be poured into a larger cavity, so as to fill the cavity.

Alternatively, adhesive gels of the present invention may be employed to bond tissues together, eg to seal incisions, tears, perforations and/or fluid or gaseous leaks in tissues. It is well-understood that suturing and stapling delicate tissue can cause tissue damage/weakness in itself, and consequential problems, eg leaks of biological fluids or bacterial infections. Bioadhesives have been described that provide means of binding tissues. However, none of these compositions have been found to be entirely satisfactory. There still exists a need for effective bioadhesive compositions that are truly safe and efficient, and whose properties can easily be tailored to suit the nature of the tissue and the extent of the damage.

Similarly, the protein gels are suitable for coating prosthetics and surgical implements, eg catheters or stents. Such a coating may have bioadhesive properties that aid retention of the device in the desired location. The use of natural proteins in the polymers, and in particular HSA, will reduce the risk of the implant being rejected by the body's natural defences against the introduction of a foreign body.

The protein polymers of the present invention are suitable for coating glass or plastic plates for diagnosis (eg ELISA, ELISPOT) or processing purposes, eg for use in the growing of cells, including stem cells.

Hard gels may be prepared using high levels of dicarboxylic acid spacer and/or EDC. It is envisaged that hard gels of the present invention may be used to strengthen and/or repair bone or cartilage, as artificial bone implants or other prosthetic devices. The gel may be formed in situ or pre-formed in a mould.

The invention will now be described in greater detail, by way of illustration only, with reference to the following non-limitative Examples, which demonstrate that:
Varying the conditions of the reactions in terms of component concentration and composition, pH and time can produce the different forms of the polymers.
Soluble polymers are more easily produced at neutral pH with lower protein concentrations.
Increasing the levels of spacer and activator in the reaction will produce insoluble particles, which are also produced when the soluble polymers are mixed with organic solvents.
Increasing the protein concentration and lowering the pH of the reaction produces gels. Further, it is possible to alter the physical characteristics of the gel (soft to hard and adhesive properties) by varying the ratio of the gel components, protein concentration and pH or a combination of these factors. This is an important factor in the production of gels for therapeutic uses including wound dressings, gel implants and bioadhesives.

### ABBREVIATIONS

DMSO Dimethylsulfoxide

EDC Ethyl[dimethylaminopropyl]carbodiimide

EMCH N-[maleimidocaproic acid]hydrazide

HSA Human serum albumin

PBS Phosphate buffered saline

### DESCRIPTION OF FIGURES

FIG. 1 shows the separation of a soluble polymer of the present invention by gel filtration on a Sepharose 6B column using standard conditions, wherein the absorbance is monitored at of 280 nm.

FIG. 2 shows the release of tetracycline from a gel of the present invention over a 45 hour period.

### EXAMPLE 1

### Formation of Soluble Protein Polymers

1.1 Formation of a Soluble Polymer of HSA using Sebacic Acid

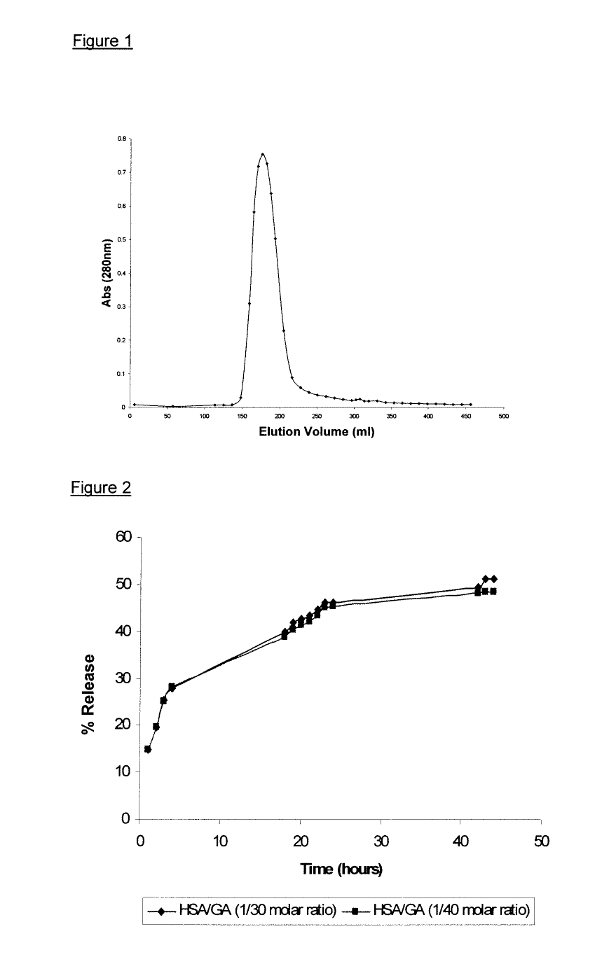

Sebacic acid (146 mg) in 2.5 ml DMSO was added to 10 ml 20% HSA solution (BPL, Zenalb) and 20 ml 0.01 M PBS buffer pH=7.4 with stirring until the solution became clear. EDC (276 mg) in 7.5 ml PBS buffer was added to the solution and stirred for 16 hours (overnight). The resulting solution was centrifuged to remove the small amount of insoluble polymer. The soluble fraction was gel-filtered on a Sepharose 6B column using standard conditions. Protein elution was monitored at A_{280 nm}. The result is shown in FIG. 1. Monomeric HSA elutes at ˜340 mls.

1.2 Preparation of a Soluble Polymer of HSA using Adipic Acid

Adipic acid 26.3 mg in 1 ml 50% ethanol was added to a stirred solution of 5 ml 20% HSA solution (BPL,Zenalb) and 25 ml 0.01 M PBS buffer, pH=7.4, with stirring until the solution became clear. EDC, 69 mg in 4 ml PBS buffer, was added dropwise to the solution with stirring. The resulting solution was stirred for a further 2 hrs. The resulting solution was centrifuged to remove the small amount of insoluble polymer. The soluble fraction was gel-filtered on a Sepharose 6B column (as in Example 1.1 above) using standard conditions.

1.3 Linking of Fibrinogen to Soluble HSA Protein Polymer to Produce a Platelet Substitute

In this example a platelet substitute (enhancer) is prepared by immobilising the clotting factor, fibrinogen, to the surface of the HSA soluble polymer in such a way as to maintain the biochemical activity of the fibrinogen. The platelet substitute can be delivered by intravenous infusion and is activated at the site of internal wounds in the blood vessels.

1.3.1 Preparation of Soluble Protein Polymer

Sebacic acid (30 mg) in 1.25 ml DMSO was added to 5ml 20% HSA solution (BPL, Zenalb) in 15 ml PBS buffer (0.01M; pH=7.4) and stirred until the solution became clear. EDC (57 mg) in 4 ml PBS buffer was added to the HSA/spacer solution and stirred at room temperature for 3 hours.

Other dicarboxylic acids of varying carbon chain length can be substituted for sebacic acid in the above reaction.

1.3.2 Thiolation of Protein Polymer

2-iminothiolane (210 mg) was added as solid to the polymer solution, followed by incubation in the dark at room temperature for 1.5 hours. The polymer was then desalted by gel filtration in 0.01 M; pH=7.4 PBS solution on a Sephadex G25 column using standard conditions.

1.3.3 Activation of Fibrinogen for Coupling to Polymer

Fibrinogen (750 mg) in 10 ml 0.05 M phosphate buffer was mixed with 2.5 ml 100 mM sodium periodate in 0.1 M sodium acetate buffer and incubated in the dark at room temperature for 30 minutes. The activated fibrinogen was then desalted in 0.01 M; pH=7.4 PBS solution by gel filtration on a Sephadex G25 column. The activated sugars were reacted with a hydrazide, in this example N-[maleimidocaproic acid]hydrazide (EMCH) (11 mg) for 2 hours in the dark at room temperature.

1.3.4 Conjugation of Activated Fibrinogen with Protein Polymer

The activated EMCH-fibrinogen solution was added to the iminothiolated polymer solution and stirred overnight. The resulting solution was centrifuged to remove any insoluble material and then gel-filtered in 0.01 M; pH=7.4 PBS solution on a Sepharose 6B column.

### EXAMPLE 2

### Formation of Insoluble Particles

2.1 Formation of an Insoluble Particle in Aqueous Solutions

Insoluble protein polymer particles can be prepared by methods analogous to those of Example 1, but with increased concentration of dicarboxylic acid spacer and EDC and/or increased reaction time whilst maintaining low protein concentration.

HSA (1 ml 20%; BPL, Zenalb) and glutaric acid were mixed in 3 ml of distilled water at a molar ratio of 1/40. EDC in 1ml distilled water was added to the stirred solution in 1/120 molar ratio HSA/EDC. The solution was stirred for 3 hours at room temperature and then centrifuged. The pellet was washed with distilled water and then dried.

2.2 Formation of an Insoluble Particle in Organic Solvents

Insoluble particles can also be produced by the dispersion of soluble polymers produced in Example 1 above into organic solvents, eg acetone.

One volume of soluble polymer solution (Example 1) was mixed with 10 volumes acetone for 15 min at room temperature. The resultant particles could be collected by centrifugation or decanting.

### EXAMPLE 3

### Formation of Protein Polymer Gels

3.1 Preparation of HSA Polymer Gel Using Sebacic Acid and High Concentration of HSA Solution

A solution of 48.5 mg sebacic acid in 1 ml DMSO was added to 4 ml HSA 20% solution (BPL, Zenalb). The solution was stirred until it became clear. A solution of 92 mg of EDC in 2 ml distilled water was added. The final concentration of HSA in the reaction was 114 mg/ml. The final molar ratio of HSA/sebacic acid/EDC was 1/20/40.

The resulting mixture formed a gel 30 seconds after addition of EDC.

It was noted that in an equivalent experiment to this example, but in the absence of the dicarboxylic acid, a gel was formed after 2 hours. The properties of the gel in this case were not suitable for wound dressings being of a hard, brittle nature that would make them difficult to apply and remove. The time to gel in situ would be too long for practical use.

3.2 Preparation of HSA Polymer Gel Using Sebacic Acid and Low Concentration of HSA Solution

The same experimental procedure was used as described in Example 3.1, except that the final concentration of HSA was 72 mg/ml. The final molar ratio of HSA/sebacic acid/EDC was 1/20/40.

The resulting mixture formed a gel in less than 5 minutes.

3.3 Preparation of HSA Polymer Gel Using Adipic Acid and High Concentration of HSA Solution

Adipic acid, 35 mg, was dissolved in 4 ml 20% HSA solution (BPL, Zenalb). A solution of 92 mg EDC in 2 ml distilled water was added as above. The final molar ratio of HSA/adipic acid/EDC was 1/20/40.

The resulting mixture formed a soft gel polymer after 2 minutes.

3.4 Preparation of Gel Containing Haemoglobin

HSA (300 mg) and haemoglobin (100 mg), sebacic acid (24.25 mg in 0.5 ml DMSO), EDC (46 mg in 1 ml distilled water) and 2 ml PBS buffer (as above) were mixed together to give a final protein concentration of 80 mg/ml.

A gel was formed after 10 minutes.

### EXAMPLE 4

### The effect of Spacer Length on Gel Characteristics

In order to determine the effects of varying the dicarboxylic acid spacer chain length, protein polymer gels were prepared using HSA at various concentrations and four different dicarboxylic acid spacers, with EDC as activator.

A solution of dicarboxylic acid in DMSO (120 μmoles in 250 μl) or (90 μmoles in 250 μl) was added to 1 ml of 20% aqueous HSA solution, in dicarboxylic acid/HSA molar ratios of 40/1 and 30/1. The solution was stirred at room temperature until it became clear. An aqueous solution of EDC was then added in EDC/dicarboxylic acid molar ratio of 2/1. The gelling time and the properties of the gels are detailed in Tables 1-3 below.

The gelling reaction is a biphasic reaction: initial gelling is followed by a secondary, “curing”, stage. Gelling time relates to initial observed gelling, and gel hardness refers to the final state of the gel after “curing”.

TABLE 1
The effect of dicarboxylic acid chain length on gelling time using
1/40/80 HSA/dicarboxylic acid/EDC molar ratio

| | |
|---|---|
| HSA | Gelling time (sec.) |

| conc. | Glutaric | Adipic acid | Suberic | Sebacic |
|---|---|---|---|---|
| (mg/ml) | acid (C5) | (C6) | acid (C8) | acid (C10) |
| 151 | 40 | 23 | 21 | 19 |
| 140 | 42 | 25 | 23 | 22 |
| 127 | 49 | 27 | 27 | 24 |
| 108 | 90 | 45 | 30 | 25 |
| 93 | 130 | 58 | 42 | 30 |

TABLE 2
The effect of dicarboxylic acid chain length on gelling time using
1/30/60 HSA/dicarboxylic acid/EDC molar ratio

| HSA | Gelling time (sec.) | | | |
|---|---|---|---|---|
| conc. | Glutaric acid | Adipic Acid | Suberic acid | Sebacic acid |
| (mg/ml) | (C5) | (C6) | (C8) | (C10) |
| 151 | 46 | 31 | 29 | 23 |
| 140 | 60 | 35 | 33 | 26 |
| 127 | 75 | 43 | 38 | 32 |
| 108 | 140 | 60 | 45 | 37 |
| 93 | 240 | 95 | 62 | 50 |

TABLE 3
The effect of HSA concentration and dicarboxylic acid chain length on gel
properties using 1/40/80 HSA/dicarboxylic acid/EDC molar ratio

| HSA | | | | |
|---|---|---|---|---|
| conc. | Gel Properties | | | |
| (mg/ | Glutaric acid | Adipic acid | Suberic acid | Sebacic acid |
| ml) | (C5) | (C6) | (C8) | (C10) |
| 151 | Hard rubbery | Turbid, hard, | Turbid, very | Very hard, |
| | gel, slightly | rubbery gel. | hard brittle gel | turbid, brittle |
| | turbid. | | | gel |
| 140 | Clear, hard | Turbid, hard, | Turbid, very | Very hard, |
| | rubbery gel | rubbery gel. | hard brittle gel | turbid, brittle |
| | | | | gel |
| 127 | Clear | Turbid, hard, | Turbid, hard, | Hard, white, |
| | medium/hard | rubbery gel. | slightly | brittle gel |
| | rubbery gel | | rubbery gel | |
| 108 | Very soft clear | Initially soft | Initially | Hard, white, |
| | gel | gel, hard after | medium/hard | brittle gel |
| | | 3 mins. | turbid rubbery. | |
| | | Slightly turbid | After 4 min | |
| | | | very hard, | |
| | | | white, brittle | |
| | | | gel | |
| 93 | Very soft clear | Initially soft | Initially turbid, | Hard/medium, |
| | gel | gel, medium, | soft/medium | white, brittle |
| | | brittle gel after | rubbery. After | gel |
| | | 3 mins. | 4 min very | |
| | | Slightly turbid | hard, white gel | |

At each HSA concentration the gelling time decreases and the gels become generally harder, less rubbery and more turbid with increasing dicarboxylic acid chain length. Increasing the HSA concentration decreases the gelling time and increases the hardness of the gel.

### EXAMPLE 5

### Control of Gelling Time and Gel Properties

Different applications of the gels will demand different gelling times and gel consistencies. Gels can be formed in seconds or over much longer periods. Gels can be extremely soft and “sticky” or very hard and rubbery. There are several approaches to controlling these parameters and for any application any or all of the following approaches can be used. All gels described below were clear unless otherwise stated.

5.1 Control of Gelling Time and Gel Characteristics by Varying the Molar Ratio of HSA to Dicarboxylic Acid Spacer

Gels were produced by dissolving glutaric acid (GA) in aqueous HSA solution (20% USP) at room temperature, and then adding a solution of EDC in distilled water to activate the gelling reaction. The gelling mixture was inverted gently several times to ensure complete mixing.

The molar ratio of HSA to glutaric acid was varied from 1/0 to 1/40 at two EDC concentrations. Experimental results are shown in Tables 4 and 5 below.

TABLE 4
Effect of changing HSA/GA molar ratio
(molar ratio HSA to EDC of 1:35)

| HSA/GA | Gelling time | Gel pH | Gel Properties |
|---|---|---|---|
| 1/0 | Over 2 hrs | 7.1 | Soft |
| 1/3.5 | 9 m | 6.8 | Medium |
| 1/5 | 5 m 25 s | 6.5 | Medium |
| 1/10 | 3 m 15 s | 5.6 | Soft |
| 1/20 | 3 m 40 s | | Soft gel, redissolves after 3 m |
| 1/40 | | | No gel formed |

TABLE 5
Effect of changing HSA/GA molar ratio
(molar ratio HSA to EDC of 1:70)

| HSA/GA | Gelling time | Gel pH | Gel Properties |
|---|---|---|---|
| 1/0 | About 30 m | 7.6 | Medium |
| 1/3.5 | 4 m 30 s | 7.2 | Medium-hard |
| 1/5 | 2 m 45 s | 7.1 | Medium-hard |
| 1/10 | 1 m 30 s | 6.2 | Hard |
| 1/20 | 1 m 5 s | 5.3 | Medium |
| 1/40 | 1 m 15 s | | Medium-soft gel, redissolves |
| | | | within 30 m |
| 1/50 | 1 m 45 s | | Soft gel, redissolves within 5 m |

Initially increasing the levels of glutaric acid decreases the gelling time and produces harder gels. However at higher levels of glutaric acid the gels are unstable, this can be offset by increasing the levels of EDC. This is discussed in Example 6.

5.2 Control of Gelling Time and Gel Characteristics by Varying the HSA Concentration

Gels were prepared using the method described in Example 5.1. A molar ratio of HSA to glutaric acid of 1/5 and a molar ratio of HSA to EDC of 1/70 were used. The concentration of HSA was varied from 182 mg/ml to 120 mg/ml. Results are shown in Table 6 below.

TABLE 6
Effect of HSA concentration on gelling time and gel hardness

| [HSA] mg/ml | Gelling Time | Gel Hardness | Gel pH |
|---|---|---|---|
| 182 | 2 m 27 s | Hard | 7.1 |
| 166 | 2 m 45 s | Medium-hard | 7.1 |
| 150 | 4 m 12 s | Medium | 7.2 |
| 135 | 5 m 55 s | Medium-soft | 7.3 |
| 120 | 7 m 15 s | Soft | 7.2 |

Decreasing the concentration of HSA results in longer gelling times and softer gels.

5.3 Control of Gelling Time and Gel Characteristics by Varying the Molar Ratio of HSA to EDC

Gels were produced as described in Example 5.1. A molar ratio of HSA to glutaric acid of 1/10 was used and the final concentration of HSA was 166 mg/ml. The molar ratio of HSA to EDC was varied from 1/35 to 1/80. Results are shown in Table 7 below.

TABLE 7
Effect of varying the HSA/EDC molar ratio

| HSA/EDC | | | |
|---|---|---|---|
| molar ratio | Gelling time | Gel pH | Gel Hardness |
| 1/35 | 3 m 5 s | 5.6 | Soft |
| 1/50 | 1 m 50 s | 5.9 | Medium |
| 1/60 | 1 m 32 s | 6.1 | Hard |
| 1/70 | 1 m 23 s | 6.2 | Hard |
| 1/80 | 1 m 5 s | 6.6 | Very Hard |

Table 7, and a comparison of tables 4 and 5 above, show that higher levels of EDC result in shortened gelling times and harder gels.

5.4 Control of Gelling Time and Gel Characteristics by Addition of Ethanol Glucose and Glycerol

A further important approach is to prepare an HSA “gelling solution” by initially modifying the HSA by reaction with a reagent, such as ethanol, glucose or glycerol (all of which have active —OH groups) in the presence of low concentrations of EDC. The preparation of HSA gelling solution by reaction with ethanol is described below.

5.4.1 Preparation of HSA Gelling Solution by Reaction with Ethanol

Ethanol was added dropwise to a stirred solution of 20% aqueous HSA. The solution was stirred until it became clear. Solid EDC was added to the solution (molar ratio HSA/EDC of 1/15) and stirred at room temperature for a minimum of 2 hours. Glutaric acid was dissolved in 20% aqueous HSA and stirred at room temperature for 30 minutes.

To prepare the final “gelling solution”, the modified HSA /ethanol solution was mixed with the unmodified HSA/glutaric acid solution in 1/1 volume ratio, and stirred at room temperature for 30 minutes. The final molar ratio of HSA to glutaric acid was 1/5.

This mixture or “gelling solution” was reacted with EDC to form the gel as in previous examples.

The volume ratio of ethanol/HSA Solution was varied from 1/7 to 1/14 results are shown in Table 8 below.

TABLE 8
Effect of modified HSA/Ethanol volume ratio on gelling time
and hardness of the gel

| Volume ratio | Molar | | |
|---|---|---|---|
| HSA/Ethanol | ratio | Gelling | |
| solution | HSA/EDC* | time | Gel Hardness |
| 7/1 | 1/35 | 1 m 45 s | Hard |
| 8/1 | 1/35 | 2 m 40 s | Hard |
| 10/1 | 1/35 | 1 m 50 s | Medium/Hard |
| 14/1 | 1/35 | 2 m 30 s | Soft |
| No ethanol | 1/35 | 5 m 30 s | Medium/Hard |

(*Molar ratio of HSA to EDC added in the gelling reaction)

Initial reaction of HSA with ethanol results in a general decrease in gelling time. At low levels of ethanol softer gels are produced. However, more than 10% v/v ethanol results in harder gels. No brittleness is found in these gels; despite their hardness they remain flexible.

The experiment described in Example 5.1 was repeated using ethanol/HSA gelling solution prepared as described above, with 10% v/v ethanol and HSA/glutaric acid molar ratios in the range 1/0 to 1/40. The results are shown in Tables 9 and 10 below.

TABLE 9
Effect of HSA/glutaric acid molar ratio using ethanol-modified
HSA (molar ratio HSA/EDC of 1/35)

| Molar Ratio | | | |
|---|---|---|---|
| HSA/GA | Gelling time | pH of the gel | Gel Hardness |
| 1/0 | About 30 m | 7.5 | Soft |
| 1/3.5 | 3 m | 6.7 | Medium |
| 1/5 | 1 m 50 s | 6.4 | Medium |
| 1/10 | 1 m | 5.5 | Medium-soft |
| 1/20 | 55 s | 4.8 | Very soft |
| 1/40 | | | No gel formed |

TABLE 10
Effect of HSA/glutaric acid molar ratio using ethanol-modified HSA
(molar ratio HSA/EDC of 1/70)

| Molar ratio | | | |
|---|---|---|---|
| HSA/GA | Gelling time | pH of the gel | Gel Hardness |
| 1/0 | 8 m | 7.7 | Soft |
| 1/3.5 | 1 m 30 s | 7.3 | Medium |
| 1/5 | 1 m 5 s | 6.9 | Medium-hard |
| 1/10 | 32 s | 5.6 | Hard |
| 1/20 | 25 s | 5.1 | Medium |
| 1/40 | 23 s | 4.3 | Soft |

5.4.2 Preparation of HSA Gelling Solution by Reaction with Glucose

A gelling solution was prepared as in Example 5.4.1 but replacing ethanol with glucose in a final HSA/Glucose molar ratio of 1/15 and a final HSA/Glutaric acid molar ratio of 1/5.

The gels produced were softer than similar gels with no glucose, and the gelling time was reduced.

5.4.3 Preparation of HSA Gelling Solution by Addition of Glycerol

Glycerol was added to 20% HSA solution (USP) in volume percentages of 0 to 16.7. Gels were then prepared using the method described in Example 5.1.

The addition of glycerol decreases the gelling time and was shown to slow down the drying out of the gel when left uncovered at room temperature for a period of two weeks.

5.4.4 Preparation of HSA Gelling Solution by Addition of Ethanol or Glucose Directly to Gelling Solution

If either ethanol or glucose is added directly to the HSA solution, and then used to form gels in the method described in Example 5.1, a similar but less marked effect was seen to that of Examples 5.4.1 and 5.4.2 respectively, where an HSA pre-modification step with the additives was included.

### EXAMPLE 6

### The Effect of Increasing the Level of Dicarboxylic Acid on the Stability of the Formed Gel

Increasing the levels of glutaric acid in the gelling solution has been shown to result in either the formation of initially medium to hard gels that revert to soft gels with time, or soft gels that redissolve to form viscous solutions. Control of this dissolution process could be a useful method of controlling delivery of drugs in the various applications described herein.

Gels were prepared following the method described in Example 5.1. The molar ratio of HSA to glutaric acid was varied from 1/5 to 1/35, at two molar ratios of HSA to EDC. Results are shown in Table 11 below.

As the ratio of glutaric acid increased, the gelling times decreased up to the point were no gel was formed. Intermediate levels produced gels that redissolved to form viscous solutions on standing. This was shown to be a result of pH changes during the reaction. At low levels of glutaric acid the pH of the gelling solution climbs to 6-7 after addition of EDC until the gel forms. At high levels of glutaric acid, the pH climbs initially then falls again towards an acidic pH of 5-6 causing the soft gel to redissolve or preventing a gel from forming.

TABLE 11
Effect of glutaric acid level on the stability of gel formed

| HSA/GA/EDC | | |
|---|---|---|
| molar ratio | Gelling time | Gel properties |
| 1/5/35 | 9 min 20 sec. | Clear medium to soft gel |
| 1/10/35 | 5 min 25 sec. | Soft gel becomes viscous solution |
| | | after 10 min. |
| 1/15/35 | — | No gel formed. |
| 1/10/50 | 3 min | Very soft gel becoming very hard |
| | | after 5-25 min, reverting to a |
| | | medium gel overnight. |
| 1/15/50 | 2 min 45 sec. | Very soft gel becoming very hard in |
| | | 4-8 min, reverting to a soft gel |
| | | overnight |
| 1/20/50 | 2 min 30 sec. | Soft gel becoming medium to hard |
| | | in 4 min, forming a viscous solution |
| | | after 1 hr. |
| 1/25/50 | 2 min 30 sec. | Soft gel becoming medium after 4 min, |
| | | forming a viscous solution after |
| | | 30 min. |
| 1/30/50 | 3 min | Very soft gel becoming viscous |
| | | solution after 7 min. |
| 1/35/50 | — | No gel formed |

### EXAMPLE 7

### Controlling Gel pH

The gelling reaction is best performed at acidic pH. It is possible to raise the pH of the final gel to close to physiological pH. There are two ways of controlling gel pH. One approach is to vary the molar ratio of HSA to the dicarboxylic acid; low levels of dicarboxylic acid give gels of close to physiological pH. The second approach is to vary the molar ratio of HSA to EDC, with high EDC levels resulting in gels of higher pH. For those skilled in the art it can be seen that it is possible to find a balance of conditions that achieves the required gel consistency for a particular application at the desired pH.

7.1 Controlling pH of Gels Using Different Concentrations of Dicarboxylic Acids

Gels were prepared by dissolving glutaric acid in 20% HSA solution (USP) and adding a solution of EDC in distilled water to give a final HSA concentration of 166 mg/ml. Molar ratios of HSA to EDC of 1:35 and 1:70 were used. The results are shown in Tables 4 and 5 above.

At these EDC levels, gels can be formed using a molar ratio of HSA to glutaric acid of 1:20 or less. At higher levels of dicarboxylic acid the gels are unstable if they form at all, as discussed in Example 6. Gel pH values in the range of 5.3 to 7.6 were obtained.

7.2 pH Control of Gels Using Varying Levels of EDC

Gels were prepared by dissolving glutaric acid in 20% HSA solution (USP) at a HSA to glutaric acid molar ratio of 1:10. Solutions of EDC in distilled water were added to give a final concentration of HSA of 166 mg/ml and molar ratios of HSA to EDC of 1:35 to 1:80. Results are shown in Table 6 above.

Gel pH values in the range of 5.6 to 6.6 were achieved by varying the levels of EDC. This is also supported by a comparison of the data in Tables 4 and 5 above. Increasing the levels of EDC in the gelling mixture also results in shorter gelling times and harder gels.

### EXAMPLE 8

### Production of a Bioadhesive

The bioadhesive gel was prepared either as a liquid or a dry powder. The tensile strength was measured by applying the liquid or powder between two pieces of meat (3 cm² beefsteak). One piece of meat was attached to card and could be held in place by a clamp and stand. Weights were attached to the second lower piece of meat to measure the tensile strength. The meat was incubated at 37° C. for 5 minutes prior to addition of weights.

8.1 HSA (4 ml 20%; BPL, Zenalb) was Mixed with Glutaric Acid and EDC at a Ratio of 1/50/100 Respectively.

The measured tensile strength was 63 mg/mm².

8.2 A Dry Powder Formulation was Prepared by Mixing 200 mg Freeze Dried HSA (Sigma) with Glutaric Acid and EDC in a Molar Ratio of either 1/50/100 or 1/60/140 Respectively.

The tensile strength increased with an increase in ratio of spacer and EDC. The 1/50/100 blend gave a tensile strength of ˜180 mg/mm². The 1/60/120 blend gave a tensile strength of ˜280 mg/mm².

### EXAMPLE 9

### Release of Drugs from Gel (Tetracycline)

To 1 ml 20% HSA solution was added 150 μl of a 10 mg/ml solution of tetracycline in ethanol. Gels were formed as described in previous examples above using molar ratios of HSA/glutaric acid/EDC of 1/30/60 and 1/40/80 respectively. The gel was left overnight before being placed in a vial containing 5 ml distilled water. The release of tetracycline with time was measured at 364 nm (FIG. 2).

### EXAMPLE 10

### Stability of HSA Gelling Solutions

10.1 Stability of Ethanol-Modified HSA Gelling Solution at 4° C. and Room Temperature

Ethanol-modified HSA gelling solution (prepared as described in Example 5.4.1) was sterile filtered through a 0.22 μm filter. Half of the solution was stored at 4° C. and half at room temperature, in sealed vials. On days 0, 7, 21 and 28, aliquots of the solutions were reacted with aqueous EDC solution, and the gelling time, gel characteristics, pH and gel stability were compared.

TABLE 12
Storage of gelling solution at 4° C.

| Day | Gelling Time | Gel Characteristics |
|---|---|---|
| 0 | 2 min 10 sec | Clear medium/hard gel |
| 7 | 2 min 15 sec | Clear medium/hard gel |
| 21 | 2 min | Clear medium/hard gel |
| 28 | 2 min 15 sec | Clear medium/hard gel |

All gels prepared were held in sealed vials at 37° C. for 14 days, to compare gel stability; none showed any sign of deterioration or bacterial growth in this period.

TABLE 13
Storage of gelling solution at room temperature

| Day | Gelling Time | Gel Characteristics | Gel pH |
|---|---|---|---|
| 0 | 2 min 10 sec | Clear medium/hard gel | 6.9 |
| 7 | 2 min 10 sec | Clear medium/hard gel | 6.9 |
| 21 | 2 min | Clear medium/hard gel | 7.0 |
| 28 | 2 min 10 sec | Clear medium/hard gel | 6.9 |

This data demonstrates that the ethanol-modified HSA gelling solution is stable for at least 4 weeks at 4° C. and at room temperature.

10.2 Stability of Glucose-Modified HSA Gelling Solution at 4° C. and at Room Temperature

The above experiment was repeated using glucose-modified HSA gelling solution (as described in Example 5.4.2). The solution stored at room temperature was shown to be stable for 2 weeks. The solution stored at 4° C. was shown to be stable for at least 4 weeks.

10.3 Stability of HSA/Glutaric Acid Solution at 4° C. and at Room Temperature

A solution of HSA (200 mg/ml) and glutaric acid (molar ratio HSA/glutaric acid of 1/37) was shown to be stable for at least 4 weeks at 4° C. and at room temperature, using the procedure described in Example 10.1.

10.4 Stability of HSA/Adipic Acid Solution at 37° C. and at Room Temperature

A solution of HSA (200 mg/ml) and adipic acid (molar ratio HSA/adipic acid of 1/30) was shown to be stable for at least 3 weeks at 37° C. and at room temperature, using the procedure described in Example 10.1.

### EXAMPLE 11

### Stability of Formed Gels

Gels with a molar ratio HSA/glutaric acid/EDC of 1/40/80, 1/50/100, 1/60/120 and 1/70/140 were held at 4° C., room temperature and 37° C. in sealed vials for a 6 week period. All gels stored at 4° C. and room temperature were stable for 6 weeks, although the turbidity of the higher ratio gels increased slightly after 4 weeks. All gels stored at 37° C. were stable for 2 weeks. By 3 weeks these gels had increased in hardness and had become more turbid. None of the gels showed any signs of bacterial growth.

### EXAMPLE 12

### In Situ Application of Gel

Wells (2 cm² and 0.5 cm deep) were cut into pieces of pig skin in vitro. Gels (prepared as described in Example 5.1 above) were formed in situ in the wells, covered with a vapour permeable membrane (eg Tagaderm, 3M) and incubated at 37° C. The gels remained soft and did not dry out. They were easily removed from the “wound” attached to the membrane.

1-63. (canceled)

64. A method of forming a wound dressing, which method comprises forming a protein polymer by reacting a protein with an alkylene dicarboxylic acid spacer of the formula
HOOC(CH₂)ₙCOOH
in which n is from 3 to 8, or an activated derivative thereof.

65. A method as claimed in claim 64, wherein the protein polymer is formed in situ.

66. A method as claimed in claim 64, wherein the protein polymer is formed prior to application.

67. A method as claimed in claim 66, wherein a supporting substrate is incorporated into the dressing.

68. A method as claimed in claim 64, further comprising the application to the wound dressing of a vapour-permeable membrane.

69. A method as claimed in claim 64, wherein the protein is albumin.

70. A method as claimed in claim 69, wherein the albumin is human serum albumin.

71. A method as claimed in claim 64, wherein the protein is a recombinant product.

72. A method as claimed in claim 64, wherein the spacer is activated to facilitate reaction with the protein molecules.

73. A method as claimed in claim 72, wherein the spacer is activated with a carbodiimide compound.

74. A method as claimed in claim 72, wherein the spacer is activated with ethyl[dimethylaminopropyl]-carbodiimide.

75. A wound dressing comprising a protein polymer formed by reacting a protein with an alkylene dicarboxylic acid spacer of the formula
HOOC(CH₂)ₙCOOH
in which n is from 3 to 8, or an activated derivative thereof.

76. A wound dressing as claimed in claim 75, which comprises a bandage impregnated with the protein polymer.

77. A wound dressing as claimed in claim 75, which is in the form of a gel sheet.

78. A wound dressing as claimed in claim 77, in which the gel sheet has a supporting substrate.

79. A wound dressing as claimed in claim 75, which further comprises one or more therapeutically active agents.

80. A wound dressing as claimed in claim 79, wherein the therapeutically active agents are selected from the group consisting of antibiotics, antivirals, anti-inflammatory agents, pain killers, haemostatic agents, phages, growth factors, anti-scarring agents, odour-absorbing agents, and agents that promote angiogenesis.

81. A method of forming a protein polymer, which method comprises reacting albumin with an alkylene dicarboxylic acid spacer of the formula
HOOC(CH₂)ₙCOOH
in which n is from 3 to 8, or an activated derivative thereof.

82. A method as claimed in claim 81, wherein the protein is human serum albumin.

83. A method as claimed in claim 81, wherein the dicarboxylic acid is activated with a carbodiimide activating agent.

84. A method as claimed in claim 83, wherein the dicarboxylic acid is activated with ethyl[dimethylaminopropypl]-carbodiimide.

85. A protein polymer formed by reacting albumin with an alkylene dicarboxylic acid spacer of the formula
HOOC(CH₂)ₙCOOH
in which n is from 3 to 8, or an activated derivative thereof.

86. A protein polymer as claimed in claim 85, which is in the form of a solution.

87. A protein polymer as claimed in claim 85, which is in the form of insoluble particles.

88. A protein polymer as claimed in claim 85, which is in the form of a gel.

89. A protein polymer as claimed in claim 85, wherein the protein polymer is conjugated with one or more clotting agents or active peptide derivatives.

90. A protein polymer as claimed in claim 85, which polymer is conjugated to a therapeutically active agent, or a precursor thereof, or to a contrast agent, and to a targeting moiety having an affinity with a specific locus within the body.

91. A kit for the preparation of a wound dressing according to claim 75, which kit comprises a first composition and a second composition, the first composition and the second composition being held in separate containers such that reaction between the first composition and the second composition is prevented.

92. A method of treatment of the human or animal body, which method comprises the administration to the body of a protein polymer as claimed in claim 85.

93. A method as claimed in claim 92, wherein the protein polymer is administered topically.

94. A method as claimed in claim 92, wherein the protein polymer is administered in the form of a solution.

95. A method as claimed in claim 93, wherein the protein polymer is administered in the form of a powder.

96. A method as claimed in claim 93, wherein the protein polymer is administered in the form of a gel.

97. A method as claimed in claim 93, wherein the albumin and the dicarboxylic acid spacer are administered to the body, such that the protein polymer is formed in situ.
